(19) United States
(12) Patent Application Publication
(10) Pub. No.: US 2019/0136171 A1
(43) Pub. Date: May 9, 2019

(54) CELL CULTURE CONTAINER AND CELL CULTURE METHOD USING THE CONTAINER

(71) Applicants: SHIMADZU CORPORATION, Kyoto-shi (JP); KYOTO UNIVERSITY, Kyoto-shi (JP); WASEDA UNIVERSITY, Tokyo (JP)

(72) Inventors: Tatsuya Munaka, Kyoto-shi (JP); Hirohisa Abe, Kyoto-shi (JP); Masaki Kanai, Kyoto-shi (JP); Masakazu Akechi, Kyoto-shi (JP); Taira Maekawa, Kyoto-shi (JP); Shinya Kimura, Kyoto-shi (JP); Eishi Ashihara, Kyoto-shi (JP); Shuichi Shoji, Tokyo (JP); Kentaro Kawai, Tokyo (JP)

(73) Assignees: SHIMADZU CORPORATION, Kyoto-shi (JP); KYOTO UNIVERSITY, Kyoto-shi (JP); WASEDA UNIVERSITY, Tokyo (JP)

(21) Appl. No.: 16/235,432

(22) Filed: Dec. 28, 2018

Related U.S. Application Data
(62) Division of application No. 13/821,063, filed on Mar. 6, 2013.

(30) Foreign Application Priority Data
Sep. 8, 2010 (JP) 2010-200679

(51) Int. Cl. C12M 1/04; C12M 1/12; C12M 1/00; C12N 5/074; C12M 1/32

(52) U.S. Cl. CPC C12M 23/24; C12M 23/04; C12M 23/12; C12N 5/0607; C12M 29/00

(57) ABSTRACT

A well has a pair of side surfaces, and one of the side surfaces is in contact with a compartment of a first channel with a gas-permeable membrane being interposed therebetween and the other side surface is in contact with a compartment of a second channel with a gas-permeable membrane being interposed therebetween. The well is filled with a liquid cell culture medium, and in such a state, a high-concentration gas and a low-concentration gas, which are different in the concentration of a specific component from each other, are allowed to flow through the first and second channels, respectively, to form a concentration distribution of the specific component in the well.

### CROSS-REFERENCE TO RELATED APPLICATIONS

The present patent application is a Divisional of U.S. patent application Ser. No. 13/821,063, filed Mar. 6, 2013, which is a National Phase of International application No. PCT/JP2011/065662, filed Jul. 8, 2011, and claims priority from Japanese Application No. 2010-200679, filed Sep. 8, 2010, all of which are incorporated herein by reference in their entireties.

### TECHNICAL FIELD

The present invention relates to a cell culture container that has a well for cell culture and is intended to create an environment suitable for cell culture in the well and a cell culture method using the container. Such a cell culture container is used to, for example, simulate an in-vivo microenvironment to analyze the functions of cells or the efficacy of a drug against cells.

### BACKGROUND ART

In the living body, oxygen and nutrition are sufficiently supplied to normal tissues through blood vessels. On the other hand, it is said that tumor tissues are under low-oxygen and low-nutrient conditions because vascularization does not occur in proportion to the growth of cancer cells or each vascular structure is disordered and fragile. Such a relation between cancer and low oxygen and nutrient concentrations has been discussed, but recently has been particularly actively studied. For example, it has hitherto been found that some cancer cells have become resistant to low-oxygen conditions (see, for example, Non-Patent Document 1).

Further, Non-Patent Document 1 states that one of important factors for stem cell niche is low oxygen concentration and cancer cells are transformed into cancer stem-like cells by low oxygen concentration. As described above, as one of approaches to examine the functions of cancer closely, culture of cancer cells under low-oxygen conditions has become important, and therefore, an environment in which cancer cells are present needs to be re-created.

Currently, cell culture is generally performed in an incubator capable of maintaining conditions of 37° C. and about 5% CO₂ in a water vapor-saturated atmosphere. The concentration of oxygen in the incubator is about 20% that is almost the same as that in the atmosphere. In the case of cell culture performed under low-oxygen conditions for the purpose of research, a special incubator is used which is capable of maintaining low-oxygen conditions where the concentration of oxygen is, for example, 0.7%, or approximately 5%.

However, it is considered that there is a concentration gradient of oxygen in a microenvironment around cancer cells distant from blood vessels or around a cancer cell population that is a clump of, for example, 10 or more cancer cells having a certain size. Particularly, it is considered that, in a direction from the inside of bone marrow toward a bone, the concentration of oxygen in the deep part of the bone is almost 0%, and therefore, there is a concentration gradient of oxygen in the same direction. Therefore, in order to culture cancer cells, it is necessary to create an environment in which a concentration gradient of oxygen is present in a low-oxygen area.

However, conventional containers such as petri dishes, flasks, and well plates generally used for cell culture are too large in capacity to form a concentration gradient of oxygen. Further, a carbon dioxide incubator, a microscopic illumination lamp, a fluorescent lamp, and other electric devices are present as heat sources, and therefore, movement of a liquid (in this case, a culture medium) is caused mainly by heat convection, which makes it impossible to stably create an environment having a concentration gradient of oxygen.

On the other hand, in a μTAS (micro Total Analysis System) or a research area called microfluidics, a microcontainer (with a capacity of, for example, 1 μL or less) produced using a microfabrication technique is used. It is considered that when a liquid is contained in such a container having a very small capacity, the liquid is trapped in a closed microspace and greatly influenced by surrounding walls and is therefore less likely to flow, which suppresses the occurrence of convection even when heat sources are present.

Patent Document 1: JP 2004-508571

Non-Patent Document 1: Jikken Igaku (Experimental Medicine), Vol. 25, No. 14, p. 2139-2143, 2007

Non-Patent Document 2: Shimadzu Review 66 [1⋅2], 37-44 (2009. 9)

### DISCLOSURE OF THE INVENTION

### Problem to be Solved by the Invention

As a method for forming a concentration gradient of a specific component using such a microcontainer as described above, a method described in Patent Document 1, for example, is generally used in which two or more liquids different in concentration are allowed to flow to be brought into contact with each other. However, when such a method is applied to cell culture, a liquid cell culture medium always needs to flow, which causes a problem that cells are subjected to some kind of stress due to the stream of the liquid cell culture medium, or a problem that a liquid factor in a microenvironment around cells is carried away by the stream of the liquid cell culture medium so that the microenvironment becomes different from an actual environment around cells.

Further, the upper end of a well of the microcontainer is often made of PDMS (polydimethylsiloxane). PDMS is permeable to gas, and is therefore effective in maintaining the partial pressure of each gas in the well at the same level as in an incubator as long as the microcontainer is used for normal cell culture. However, equilibrium between the inside of the well and the inside of the incubator is achieved by the PDMS film, which makes it difficult to form a concentration gradient such as an oxygen concentration gradient in a low-oxygen area.

For the above reason, it is impossible to create an environment, in which a concentration gradient of oxygen is present in a low-oxygen area, in a state where a liquid cell culture medium remains at rest. Therefore, it is also difficult to simulate a microenvironment around tumor, culture cells that behave like cancer stem cells stably and reproducibly, and take out cancer cells that have transformed into cancer stem-like cells. Further, it is impossible to simulate a microenvironment in bone marrow for the study of leukemia because a concentration gradient of oxygen cannot be maintained in a low-oxygen area.

It is therefore an object of the present invention to make it possible to stably create an environment having a region in which the concentration of a gas typified by oxygen or carbon dioxide is suitable for cell culture.

### Means for Solving the Problem

The present invention is directed to a cell culture container comprising: a well formed inside a substrate, connected to a channel through which a liquid cell culture medium flows, and having a space that holds a cell and has a pair of opposed side surfaces, one of which is constituted by a first gas-permeable membrane that is permeable to gas but not permeable to liquid, and the other of which is constituted by a second gas-permeable membrane that is permeable to gas but not permeable to liquid; a first channel which is in contact with the well with the first gas-permeable membrane being interposed therebetween and through which a gas containing a specific component flows; and a second channel which is in contact with the well with the second gas-permeable membrane being interposed therebetween and through which a gas containing no specific component or containing the specific component at a lower concentration than the gas allowed to flow through the first channel flows.

Consideration is given to the volume of the well of the cell culture container according to the present invention. In this well, an in-vivo microenvironment is preferably simulated on an unchanged scale. Here, a cell is regarded as the smallest unit for measuring the functions of cells or tissues. When a cell is approximately regarded as a sphere having a diameter of R, the well needs to have a lower surface on which two cells can be placed to observe the interaction between two cells, and therefore, the lower surface has a size at least equal to the size of a circle having a diameter of 2R. When it is assumed that the diameter of a cell is 10 μm, the lower surface of the well needs to have a size at least equal to the size of a circle having a diameter of 20 μm. When it is assumed that the well needs to have a height twice as large as a cell, the minimum volume of the well is 6.3×10⁻⁶ mm³.

On the other hand, the maximum volume of the well is considered as about 1 mm³ (1 mm×1 mm×1 mm). If the volume of the well exceeds it, it is impossible to observe the interaction between cells because the cells are too distant from each other.

For the above reason, the well preferably has a capacity in the range of 6.3×10⁻⁶ mm³ to 1 mm³.

Even when the well has the above maximum volume, a liquid is trapped in a closed microspace and greatly influenced by surrounding walls and is therefore less likely to flow, which suppresses the occurrence of convection. In fact, an experiment is performed in a state where a liquid culture medium remains at rest, and for example, when the liquid culture medium is replaced, its flow rate is considered to be about 10 μm/sec at most. Even when a liquid flows at an extraordinary flow rate of 10 mm/sec, it is considered that the Reynolds number (Re) thereof is sufficiently smaller than 2300 at which the transition from laminar flow to turbulent flow occurs, and therefore laminar flow occurs and mixing of the liquid in the well is dominated by diffusion. That is, an environment created in the well is stably maintained.

It is preferred that at least one of the upper and lower end sealing members that seal the upper and lower ends of the well is a transparent window through which the inside of the well is visible from the outside. This makes it possible to confirm the position or appearance of a cultured cell from the outside with the use of a microscope or the like.

In this case, an oxygen monitoring substance whose optical properties change depending on the concentration of oxygen in a contact liquid can be fixed onto the inner surface of the transparent window. This makes it possible to optically measure a concentration distribution of oxygen formed in the well. Such a cell culture container can also be used as a test chip for use in verification of concentration gradient of oxygen in the well.

As the “oxygen monitoring substance”, for example, a fluorochrome such as platinum porphyrin can be used. The measurement of concentration distribution of oxygen using platinum porphyrin or the like will be described in detail with reference to an embodiment.

The present invention is also directed to a cell culture method for culturing a cell using the cell culture container according to the present invention whose at least one of upper and lower end sealing members that seal the upper and lower ends of the well formed in a substrate is a transparent window through which an inside of the well is visible from the outside, the method including the steps of: filling the well with a liquid cell culture medium and introducing a cell into the well; observing the inside of the well through the transparent window to recognize a position where the cell introduced into the well stays; setting flow rates of gases allowed to flow through the first and second channels based on a previously-determined relationship between flow rates of gases allowed to flow through the first and second channels and a concentration distribution of a specific component formed in the well to control the concentration of the specific component at the position where the cell stays to a desired level; and allowing the gases to flow at the flow rates set in the gas flow rate setting step through the first and second channels while the liquid cell culture medium in the well remains at rest.

An example of the specific component whose concentration gradient is formed in the well is oxygen. In this case, the concentration of oxygen in the well is preferably less than 21%. This makes it possible to form, in the well, a concentration gradient of oxygen close to that in an in-vivo environment.

An example of a cell to be cultured by the cell culture method according to the present invention is an iPS cell. In this case, it is preferred that the flow rates of a high-concentration gas and a low-concentration gas are adjusted so that the concentration of oxygen at the position where the cell stays becomes about 5%.

Some cancer cells are preferably cultured under low-oxygen conditions. Here, the low-oxygen conditions are conditions where the concentration of oxygen is almost 0 to 5%. The reason for this is as follows: An environment in lung alveoli is richest in oxygen in the human body, and the partial pressure of oxygen in lung alveoli is 100 mmHg at one atmospheric pressure, that is, the concentration of oxygen therein is 100/760=13%. Oxygen in lung alveoli is carried by hemoglobin and transported around the body through blood vessels, and the partial pressure of oxygen in the terminals of capillary vessels is 45 to 50 mmHg, that is, the concentration of oxygen therein is 5.9 to 6.5%. The partial pressure of oxygen in stromata in normal tissues such as nerves, collagen fibers, and fibroblasts is 20 to 40 mmHg, that is, the concentration of oxygen therein is 2.6 to 5.2%. The concentration of oxygen in tumor is distributed in the range of 0 to 5%, and its median is about 1.3%. On the other hand, the growth and differentiation of stem cells/precursor cells are often controlled at an oxygen concentration in the range of 1 to 5%, and it has been reported that the ability of iPS cells to proliferate is enhanced at an oxygen concentration of 5%. Therefore, an appropriate concentration of oxygen under “low-oxygen conditions” for culture of cancer cells in a simulated body environment is about 0 to 5%.

### Effects of the Invention

In the cell culture container according to the present invention, a pair of opposed side surfaces of a space inside the well are constituted by first and second gas-permeable membranes permeable to gas but not permeable to liquid, and the first channel, through which a gas containing a specific component flows, is in contact with the well with the first gas-permeable membrane being interposed therebetween, and the second channel, through which a gas containing no specific component or containing the specific component at a lower concentration than the gas allowed to flow through the first channel flows, is in contact with the well with the second gas-permeable membrane being interposed therebetween, and therefore a concentration gradient of the specific component can be stably formed in a liquid cell culture medium in the well by allowing the gases to flow through the first and second channels at constant flow rates, respectively. The concentration gradient of the specific component formed in the liquid cell culture medium in the well can be controlled by adjusting the concentrations of the specific component in the gases allowed to flow through the first and second channels and the flow rates of the gases.

The cell culture method according to the present invention uses the cell culture container according to the present invention configured to allow the inside of the well to be visible from the outside to recognize the position of a cell introduced into the well to adjust the concentration of a specific component at the position of the cell to a desired level, which makes it possible to create, around the cell, an environment suitable for cell culture or an environment close to an in-vivo environment. According to this method, a high-concentration gas and a low-concentration gas always flow through the first and second channels, but a liquid cell culture medium in the well remains at rest, which makes it possible to stably create an environment suitable for culturing a cell without subjecting the cell to unnecessary stress.

### BRIEF DESCRIPTION OF THE DRAWINGS

FIG. 1A is a plan view of one embodiment of a cell culture container.

FIG. 1B is a sectional view taken along the A-A line in FIG. 1A.

FIG. 2 is a perspective view of the same embodiment.

FIG. 3 is a perspective view of a main part of a container for simulating the concentration distribution of oxygen in a well.

FIG. 4A is a concentration distribution pattern diagram showing the simulation result of concentration distribution of oxygen in the well.

FIG. 4B is a graph showing the concentration gradient of oxygen in the central part of the well.

FIG. 5 is a flow chart of a cell culture method using the cell culture container according to the same embodiment.

FIG. 6 is a graph showing one example of results of plotting an oxygen partial pressure and a measured amount of fluorescence based on Stern-Bolmer equation.

### MODE FOR CARRYING OUT THE INVENTION

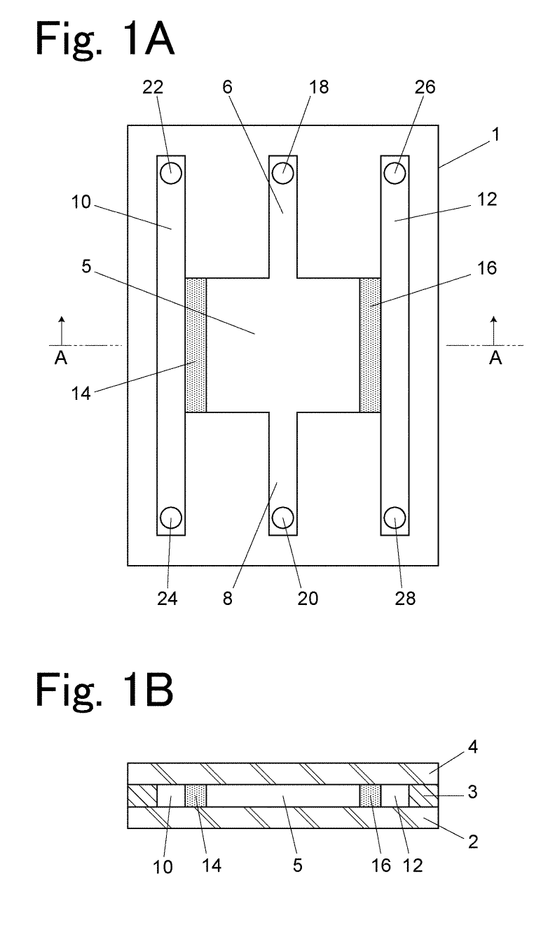

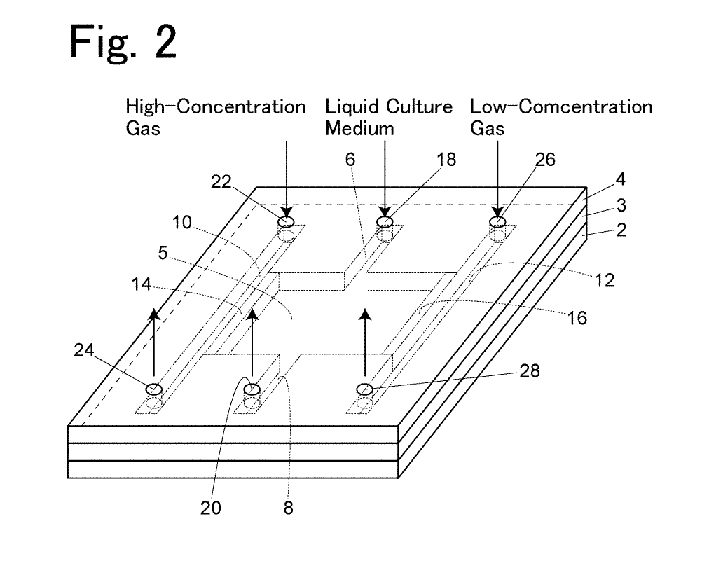

An embodiment of a cell culture container will be described with reference to FIGS. 1 and 2.

A cell culture container 1 according to this embodiment includes a well 5, a first channel 10, and a second channel 12 provided inside thereof. The well 5 is a rectangular parallelepiped space and has a pair of opposed side surfaces, one of which is in contact with a compartment of the first channel 10 with a gas-permeable membrane 14 being interposed therebetween, and the other of which is in contact with a compartment of the second channel 12 with a gas-permeable membrane 16 being interposed therebetween. The gas-permeable membranes 14 and 16 are membranes permeable to gas but not permeable to liquid. The well 5 has another pair of side surfaces, one of which is provided with a channel 6 for introducing a liquid cell culture medium, and the other of which is provided with a discharge channel 8.

The cell culture container 1 is formed by integrating two transparent substrates 2 and 4 provided as sealing substrates and a channel-forming sheet 3 interposed between the transparent substrates 2 and 4 having a through groove constituting the well 5, the first channel 10, and the second channel 12 into a single chip by thermocompression bonding. As the transparent substrates 2 and 4, flat plates such as glass substrates or quartz glass substrates can be used which are transparent to such an extent that the inside of the well 5 is visible from the outside and are not permeable to gas and liquid. The transparent substrate 4 preferably has a thickness of about 0.5 to 1.0 mm to ensure strength. The transparent substrate 2 preferably has a thickness of about 0.17 mm when it is assumed that the inside of the well 5 is observed with, for example, an invert microscope.

As a material of the channel-forming sheet 3, Neoflon (trademark) EFEP (ethylene-perfluoroethylenepropene copolymer) that is an adhesive fluorocarbon resin can be used. An appropriate thickness of the channel-forming sheet 3 is in the range of 20 μm to 1000 μm. Neoflon can be patterned by cutting. As the gas-permeable membrane 14 serving as a partition between the well 5 and the first channel 10 and the gas-permeable membrane 16 serving as a partition between the well 5 and the second channel 12, Poreflon (trademark: product of Sumitomo Electric Fine Polymer Inc.) membranes that are porous membranes made of PTFE (polytetrafluoroethylene) and having a pore size of, for example, 0.1 μm can be used.

The transparent substrate 4 that seals the upper ends of the well 5, the first channel 10, and the second channel 12 has, at positions corresponding to the ends of the introduction channel 6, the discharge channel 8, the first channel 10, and the second channel 12, through holes 18, 20, 22, 24, 26, and 28 that serve as inlets or outlets of the channels 6, 8, 10, and 12.

The cell culture container 1 can form a concentration distribution of a specific component in the well 5 by allowing a high-concentration gas and a low-concentration gas, which are different in the concentration of the specific component from each other, to flow through the first channel 10 and the second channel 12 in a state where the well 5 is filled with a liquid cell culture medium, a cell is contained in the liquid cell culture medium, and the liquid cell culture medium in the well 5 remains at rest. The high-concentration gas refers to a gas containing a higher concentration of the specific component, and the low-concentration gas refers to a gas containing a lower concentration of the specific component. The specific component is, for example, oxygen or carbon dioxide. In this embodiment, as shown in FIG. 2, the through hole 22 serves as an inlet through which the high-concentration gas is introduced into the first channel 10, the through hole 24 serves as an outlet through which the high-concentration gas is discharged, the through hole 26 serves as an inlet through which the low-concentration gas is introduced into the second channel 12, and the through hole 28 serves as an outlet through which the low-concentration gas is discharged. A concentration gradient whose maximum concentration is the concentration of the specific component in the high-concentration gas and whose minimum concentration is the concentration of the specific component in the low-concentration gas can be formed in the well 5 by allowing the high-concentration gas to flow through the first channel 10 at a constant flow rate and by allowing the low-concentration gas to flow through the second channel 12 at a constant flow rate.

The cell culture container 1 can be used as a test chip for measuring the concentration distribution of oxygen by fixing, as an oxygen monitoring substance, a fluorochrome such as platinum porphyrin onto the inner surface of the transparent substrate 2 or 4 in a portion corresponding to the well 5. As a method for visualizing the distribution of oxygen partial pressure, a pressure-sensitive paint method is conventionally known. This is a method utilizing the fact that a fluorochrome such as platinum porphyrin is quenched by oxygen and the amount of fluorescence is a function of oxygen partial pressure. Such a fluorochrome is dissolved in a solvent together with a matrix polymer to prepare a reagent, and the reagent is applied onto the inner surface of the transparent substrate 2 or 4 to have a thickness of, for example, 3 μm. More specifically, P-TMSP (poly(1-trimethylsilyl-1-propyne) that is a polymer having excellent gas permeability is selected as the matrix polymer, which makes it possible to cause a great change in the amount of fluorescence by the action of oxygen and reduce temperature dependence (see Non-Patent Document 2). The intensity of fluorescence is measured using, for example, a 405 nm violet laser as excitation light. The upper surface of the oxygen concentration visualizing test chip is irradiated with the violet laser homogenized by a diffuser, and fluorescence of 650 nm emitted from the fluorochrome is measured by a CCD camera.

The fluorescence intensity measured by the CCD camera is subjected to data processing to obtain oxygen partial pressures at different positions to which the fluorochrome is applied. It is known that a fluorescent reagent such as platinum porphyrin is quenched by oxygen according to Stern-Bolmer equation represented by the following equation (1). It is to be noted that in the following equation (1), I represents the intensity of emission when the partial pressure of oxygen is p, Iref represents the intensity of emission when the partial pressure of oxygen is pref (usually set to an atmospheric oxygen partial pressure of 21 kPa), and A0 to A3 are fitting coefficients.

I_{ref}/I=A₀+A₁(p/p_{ref})+A₂(p/p_{ref})²+A₃(p/p_{ref})³

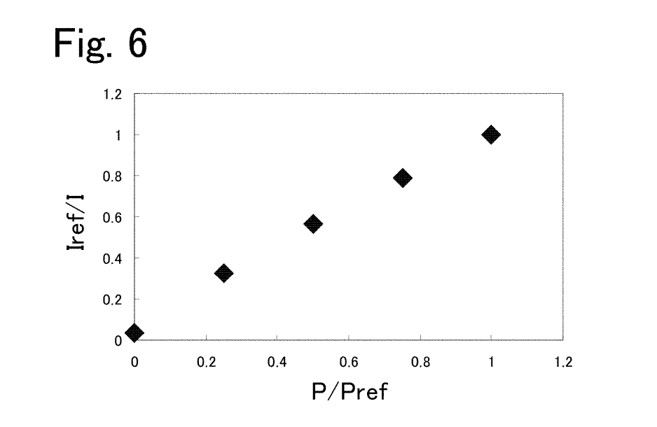

The result of plotting the oxygen partial pressure and the measured amount of fluorescence based on the above equation (1) is shown in FIG. 6. The vertical axis represents the reciprocal of the normalized amount of fluorescence and the horizontal axis represents the normalized oxygen partial pressure. As can be seen from FIG. 6, the amount of fluorescence at an oxygen partial pressure of about 0 is several tens of times larger than that at normal pressure, that is, a great change in the amount of emission is caused by a change in the partial pressure of oxygen, which indicates that the reagent offers enough performance to measure the concentration of oxygen in the well of the oxygen concentration gradient-forming chip according to the present invention.

Such a method makes it possible to visualize the concentration distribution of oxygen in the well of the test chip. This makes it possible to measure/analyze the concentration distribution of oxygen in the well of the test chip and control the concentration gradient of oxygen in the well and also makes it possible to control the concentration of oxygen at any position in the well to a desired level.

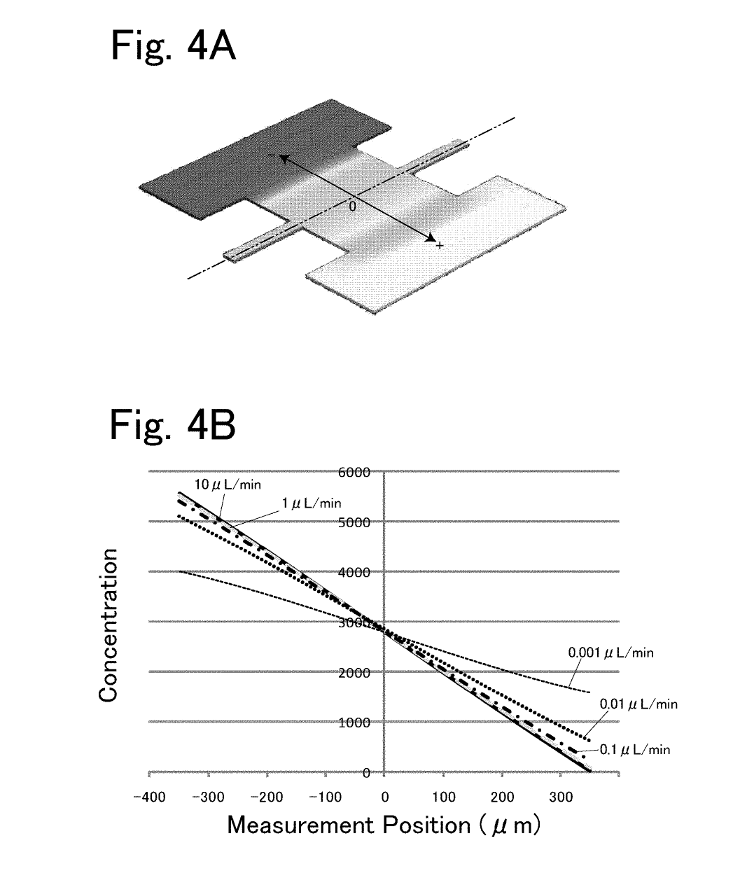

A simulation of the formation of a concentration gradient in the well 5 was performed using fluid analysis software, and the resulting data is shown in FIG. 4. As the fluid analysis software for simulation, CoventorWare (Coventor, Inc) performing analysis using a finite-element method was used. As the fluid analysis software, another analysis software using a finite-element method may also be used.

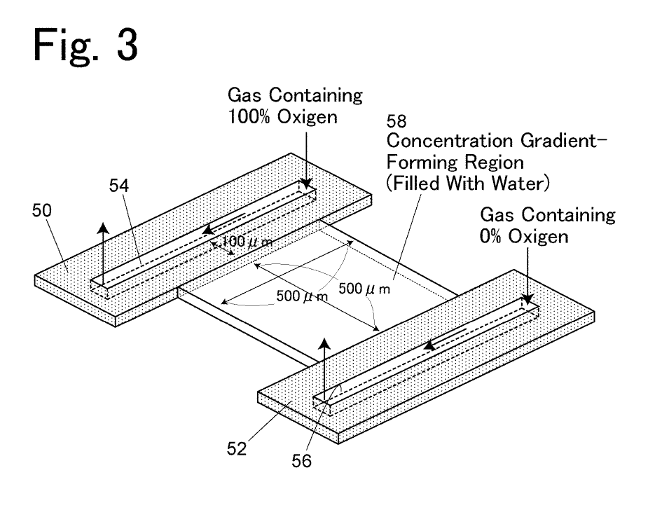

This simulation was performed using a model shown in FIG. 3 simulating the cell culture container 1. This model has a region (concentration gradient-forming region) 58 filled with water and provided between two channels 54 and 56 surrounded by porous membranes 50 and 52, respectively. The width of a compartment of each of the porous membranes 50 and 52 located between each of the channels 54 and 56 and the concentration gradient-forming region 58 is 100 μm. The concentration gradient-forming region 58 has a square planar shape with a size of 500 μm×500 μm.

In this simulation, a gas whose oxygen concentration was 100% and a gas whose oxygen concentration was 0% were allowed to flow through one of the flow channels and the other channel, respectively, at flow rates of 0.001 μL/min, 0.01 μL/min, 0.1 μL/min, 1 μL/min, and 10 μL/min to determine the concentration distribution of oxygen. FIG. 4A shows the distribution of concentration of oxygen in the model shown in FIG. 3. FIG. 4B shows the results of simulation in the central part (on the line indicated by an arrow in FIG. 4A) of the concentration gradient-forming region. As can be seen from the results, a linear concentration gradient is formed in the concentration gradient-forming region, and the concentration gradient is greater at a higher gas flow rate and is smaller at a lower gas flow rate. Therefore, the concentration gradient formed in the well 5 can be controlled and the concentration of the specific component at any position in the well 5 can also be controlled to a desired level by controlling the concentration and flow rate of the high-concentration gas allowed to flow through the first channel 10 in the cell culture container 1 and the concentration and flow rate of the low-concentration gas allowed to flow through the second channel 12 in the cell culture container 1.

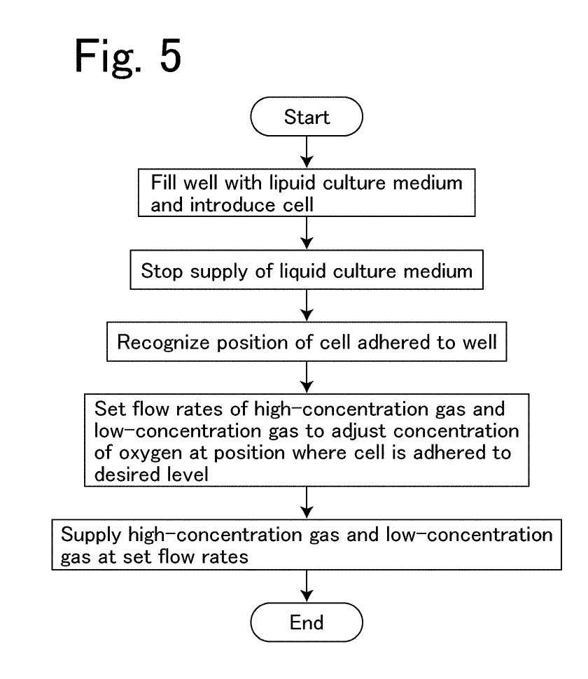

Hereinbelow, a cell culture method using the cell culture container 1 will be described with reference to FIGS. 2 and 5. Here, the cell culture method will be described with reference to a case where a cancer cell is cultured. The high-concentration gas allowed to flow through the first channel 10 is a gas containing about 5% oxygen, and the low-concentration gas allowed to flow through the second channel 12 is a gas containing 0.1% oxygen. This cell culture method is implemented on the assumption that the relationship between flow rates of the high-concentration gas and the low-concentration gas and a concentration distribution formed in the well 5 when both the gases are allowed to flow at the flow rates to reach equilibrium is previously measured or simulated, and the measured or simulated data is stored in a memory medium or the like. Further, the cell culture container 1 is in a state where the flow rates of both the gases can be set to control the concentration of oxygen at any position in the well 5 to a desired level based on the stored measured or simulated data.

Here, the measured data of the relationship between flow rates of the high-concentration gas and the low-concentration gas and a concentration distribution formed in the well 5 can be determined using, for example, a test chip having platinum porphyrin applied thereto, and the simulated data can be obtained by the simulation described above with reference to FIGS. 3 and 4.

First, the well 5 is filled with a liquid culture medium by supplying the liquid culture medium through the through hole 18 as a liquid culture medium inlet. At this time, a cancer cell is introduced into the well 5 together with the liquid culture medium. The flow rate of the liquid culture medium supplied to the well 5 is preferably controlled so that the cell stops and stays near the central part of the well 5. After the well 5 is filled with the liquid culture medium, the supply of the liquid culture medium is stopped to keep the liquid culture medium in the well 5 at rest.

The position of the cell introduced into the well 5 is recognized. An example of a method for recognizing the position of the cell is image recognition using a microscope image. The concentration of oxygen at the recognized position of the cell and conditions for creating an environment suitable for cell culture around the position of the cell are set by selecting them from the previously-prepared measured or simulated data. When the high-concentration gas and the low-concentration gas are supplied at their respective set flow rates, the concentration of oxygen in the well 5 between the first channel 10 and the second channel 12 reaches a state of equilibrium after a certain period of time and a stable concentration gradient of oxygen is formed in the well 5, which makes it possible to culture the cancer cell introduced into the well 5 under suitable conditions. The liquid culture medium in the well 5 remains at rest, and therefore, the cancer cell is not subjected to stress, and an environment around the cancer cell is kept stable. The capacity of the well 5 is 1 mm³ or less, and therefore, convection of the liquid culture medium does not occur, which makes it possible to stably maintain an environment suitable for cell culture.

It is to be noted that in this case, a gas containing about 5% oxygen is used as the high-concentration gas, but in a case where a higher concentration of oxygen is required, for example, a gas containing 21% oxygen is used as the high-concentration gas. It is believed that there is a concentration gradient of oxygen from 0% to 21% in the living body. Therefore, every environment in the living body can be re-created in the well 5 by using a gas containing 21% oxygen as the high-concentration gas.

In a case where bone marrow needs to be simulated in the well 5, an osteoblast cell, a bone-marrow cell, or an osteoclast cell may be used as the cell placed in the well 5, or in some cases, a bone fragment may be placed in the well 5. The placement of a bone fragment can be performed by, for example, placing a bone fragment in the well whose upper end is open in the process of producing the chip. The bone fragment is cut with a surgical knife or the like to a small size to fit in the well and is placed with tweezers or the like at a desired position on the bottom surface of the well. Then, the process of bonding an upper member is performed. When the end of a bone presented away from blood vessels is cut along the longitudinal direction of the bone, there is a concentration gradient of oxygen in the same direction. There is a concentration gradient of oxygen in a direction toward the blood vessels, and the concentration of oxygen is maximized at the blood vessels. However, it is considered that the concentration of oxygen in terminal organs distant from the lungs is different from an atmospheric oxygen concentration of 20% but is about 5%.

In order to study the behavior of a leukemia cell, a leukemia cell is introduced into the well and observed. When a microenvironment around another tumor is simulated, one or more cancer cells are introduced into the well 5 and placed on the low-concentration side of the well 5. When a cell is confirmed to be in the resting phase of the cell cycle or to have resistance to an anticancer drug, the cell can be identified as a cancer cell that behaves like a cancer stem cell. If a cancer stem-like cell can be detected, the cell can be taken out with, for example, optical tweezers. Further, an iPS cell may be cultured in the well 5. It is believed that an iPS cell is activated in a region where the concentration of oxygen is about 5%, and therefore, an iPS cell can be cultured in the well 5 by creating a region where the concentration of oxygen is about 5% at the position of the iPS cell.

As has been described above, various cells can be cultured by the cell culture method using the cell culture container 1. Cultured cells taken out of the cell culture container 1 can be subjected to various conventional analyses such as gene analysis.

### INDUSTRIAL APPLICABILITY

The present invention can be applied to culture of cells subjected to various analyses such as gene analysis.

### EXPLANATION OF REFERENCE NUMERALS

1 cell culture container
2, 4 transparent substrate
3 channel-forming sheet
5 well
6 introduction channel
8 discharge channel
10 first channel
12 second channel
14, 16 gas-permeable membrane (porous membrane)
18, 20, 22, 24, 26, 28 through hole

What is claimed is:

1. A method of culturing cells, using a cell culture container,
the cell culture container comprising:
a well formed inside a substrate, the well being connected to a channel through which a liquid cell culture medium flows, the well having a space for holding a cell, the space having a pair of opposed side surfaces constituted by first and second gas-permeable membranes that are permeable to gas but not permeable to liquid;
a first channel disposed in contact with the well with the first gas-permeable membrane being interposed therebetween; and
a second channel disposed in contact with the well with the second gas-permeable membrane being interposed therebetween,
the method comprising the steps of:
filling the well of the cell culture container with a liquid cell culture medium and introducing a cell into the well; and
allowing a gas, which contains a specific component, to flow through the first channel and allowing a gas, which contains no specific component or contains the specific component at a lower concentration than the gas allowed to flow through the first channel, to flow through the second channel.

2. The method according to claim 1, further comprising the step of:
determining flow rates of gases allowed to flow through the first and second channels, the flow rates enabling a concentration distribution of the specific component formed in the well to control the concentration of the specific component to a certain level at the position where the cell stays.

3. The method according to claim 2, wherein the step of allowing the gases to flow through the first and second channel includes allowing the gases to flow at the flow rates set in the gas flow rate setting step through the first and second channels, with the liquid cell culture medium remaining at rest in the well remains.

4. The method according to claim 1, wherein upper and lower ends of the well of the cell culture container are sealed with upper and lower end sealing members, and at least one of the upper and lower end sealing members includes a transparent window, enabling an inside of the well to be visible from the outside, and
wherein the method further comprises the step of observing the inside of the well through the transparent window to recognize a position where the cell introduced into the well stays.

5. The method according to claim 1, wherein the specific component is oxygen.

6. The method according to claim 1, wherein the concentration of oxygen in the well is less than 21%.

7. The method according to claim 1, wherein the cell is an iPS cell, and the concentration of oxygen at the position where the cell stays is about 5%.

8. The method according to claim 1, wherein the well of the cell culture container has a capacity in the range of 6.3×10⁻⁶ mm³ to 1 mm³.

9. The method according to claim 1, wherein the transparent window is provided on an inner surface of one of the upper and lower end sealing members, and wherein the inner surface has an oxygen monitoring substance fixed thereon, the substance has optical properties that change depending on the concentration of oxygen in a contact liquid.
